(19) United States
(12) Patent Application Publication
(10) Pub. No.: US 2007/0060759 A1
(43) Pub. Date: Mar. 15, 2007

(54) Method for the preparation of citalopram

(76) Inventors: Giovanni Cotticelli, Cernusco Sul Navialio (Milano) (IT); Gianluca Di Lernia, Buccinasco (Milano) (IT); Silvia Milanesi, Bressana Bottarone (Pavia) (IT)

Correspondence Address: PERKINS COIE LLP, P.O. BOX 2168, MENLO PARK, CA, 94026, US

(21) Appl. No.: 10/577,869

(22) PCT Filed: Oct. 22, 2004
(86) PCT No.: PCT/EP04/52626

§ 371 Date: Jun. 23, 2006

(30) Foreign Application Priority Data
Oct. 28, 2003 (EP) 03425693.3

(51) Int. Cl. C07F 3/02; C07D 311/58

(52) U.S. Cl. 549/467; 260/665 G

(57) ABSTRACT

A method for the preparation of citalopram and its pharmaceutically acceptable salts is described; it's obtained starting from 5-cyanophthalide by reaction with a mixture of 4fluorophenyl magnesium bromide and 3-dimethylaminopropyl magnesium chloride. The intermediate obtained is showed here-be-low: wherein X is an halogen, preferably chlorine or bromine, which is cyclized without any isolation.

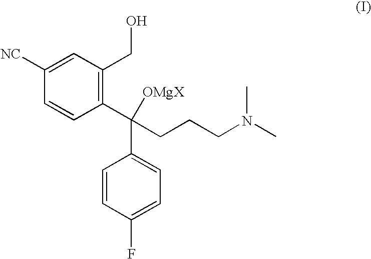

The present invention relates to a process for preparation, in just one series of reactions, of 1-[3-(dimethylamino)propyl]-1-(4-fluorophenyl)-1,3-dihydro-5-isobenzofurancarbonitrile and its pharmaceutically acceptable salts.

### BACKGROUND OF THE INVENTION

The mentioned compound, whose formula of structure is here-below reported, 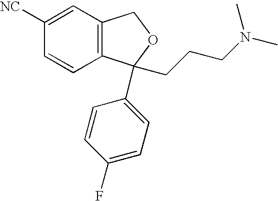
is a well known active drug, better known under its International Denomination “citalopram”, which is used in bromhydrate form for the preparation of pharmaceutical compositions for the treatment of depression.

Citalopram was first disclosed in the Belgian patent application BE-850401 (and in its equivalent US patent U.S. Pat. No. 4,136,193); several patent documents also describe methods for its preparation.

Preferably, EP-171943 describes a synthetic method with two consecutive Grignard reactions starting from 5-cyanophthalide, the first one with 4-fluorophenylmagnesium bromide, and the second one from the thus-obtained magnesium derivate (formula A), 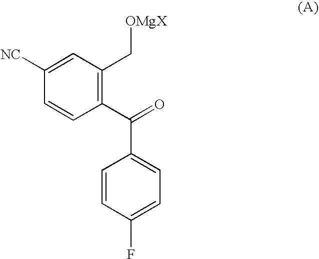
(A) with 3-(dimethylamino)propylmagnesium chloride to obtain first a magnesium intermediate (formula B) 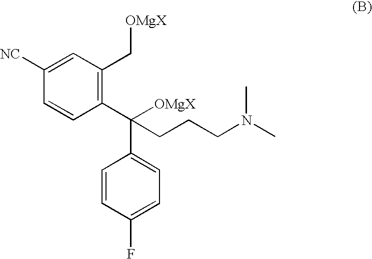
and then, after an acid hydrolysis, a diol (formula C) precursor of citalopram.

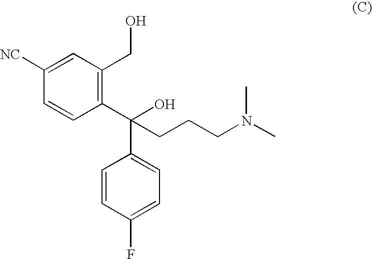

In particular, EP-171943 describes the reaction between 4-fluorophenylmagnesium bromide and 5-cyanophthalide as a slow mechanism so as to state that the intermediate of formula (A) is obtained after a slow transformation from another intermediate (A′) as reported here-below:

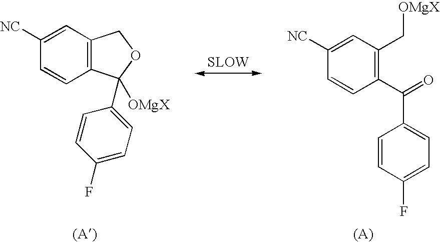

The subsequent reaction between intermediate (A) and 3-(dimethylamino)propylmagnesium chloride is described as slow too (the examples disclose a reaction time of one night).

### SUMMARY OF THE INVENTION

The present invention describes a preparation of citalopram from 5-cyanophthalide by the following route: 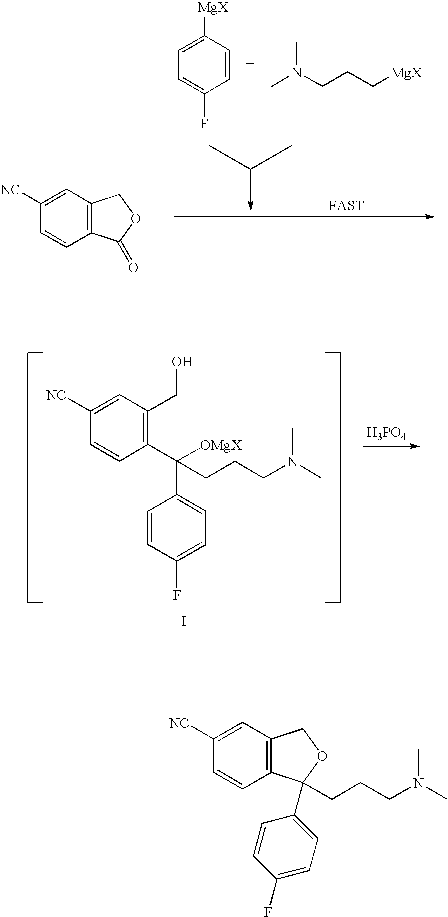
where X is an halogen, preferably chlorine or bromine.

This process is very easy and fast, it doesn't involve the formation of the above-mentioned intermediates (A), (A′) and (C) and it allows to obtain citalopram without the isolation of intermediate I.

More in details, intermediate I is synthesized from 5-cyanophthalide by adding a mixture of 4-fluorophenyl magnesium bromide and 3-dimethylaminopropyl magnesium chloride. This reaction, contrarily to EP-171943, is basically instantaneous; besides, at the end of the mixture addition, cyclization is possible by directly adding an acid (preferably orto phosphoric acid 85%) to the reaction mixture, with great operative advantages; after solvent distillation (preferably tetrahydrofuran) the reaction is finished in about two hours.

The subsequent isolation of citalopram is carried out with well known extraction methods.

The present invention relates to a novel method for the preparation of citalopram which comprises:
a) the reaction of 5-cyanophthalide with a mixture of 4-fluorophenyl magnesium halogenide and 3-dimethylaminopropyl magnesium halogenide;
b) the resulting mixture is treated with an organic or inorganic acid having a pK comprised between 0 and 3, preferably between 2 and 3.

In a preferred embodiment of the invention, 4-fluorophenyl magnesium bromide and 3-dimethylaminopropyl magnesium chloride are employed; furthermore, said acid having a pK comprised between 0 and 3 is preferably orto phosphoric acid. According to a preferred way to proceed, the process according to the invention is carried out “one pot”, without isolating the intermediate products until obtainment of citalopram.

The reaction is preferably carried out in an organic polar aprotic solvent, preferably tetrahydrofuran and/or toluene. In practice, the Grignard solution is prepared by adding a solution of 4-fluorobromobenzene in said organic polar aprotic solvent (preferably tetrahydrofuran and/or toluene) to magnesium turnings in presence of traces of iodide at solvent reflux temperature (at 50÷70° C., preferably 70° C. for tetrahydrofuran) and cooling to room temperature, after about 30 minutes. Separately a solution of ethylbromide in the same organic solvent is added to magnesium turnings in presence of traces of iodide at solvent reflux temperature; maintaining the mixture at this temperature, 3-dimethylaminopropyl chloride is added to the same solvent, reflux is maintained for further thirty minutes, then it's cooled to room temperature. The two solutions are then mixed together at room temperature.

The mixture is added to a 5-cyanophthalide suspension in the same organic polar aprotic solvent, preferably tetrahydrofuran, at −20÷+20° C., preferably at −10÷0° C. The reaction is usually finished at the end of the addition.

The acid having a pK comprised between 0 and 3, preferably orto-phosphoric acid, is then added to the reaction mixture at −10÷+20° C., preferably at 0÷+10° C.; the mixture is then heated to 55÷85° C., preferably to about 65° C., in order to distil all the tetrahydrofuran. At the end of the distillation, the mixture is kept at 60÷90° C., preferably at 70÷80° C. for 1÷3 hours, preferably for about two hours, to give citalopram.

More in general, the ring closure can be achieved both by an inorganic acid, as for instance sulfuric or phosphoric acid and by an organic acid; additionally, the ring closure can also be achieved with phosphines, preferably with triphenylphosphine. A further possibility is that of reacting the intermediate I with a labile ester forming group and then reacting the so-obtained ester with a base; preferably, the labile ester forming group is selected from the halide or the anhydride of an organic acid whereas the base is selected from triethylamine, dimethylaniline or pyridine. The halide of the organic acid may be that of methanesulfonic, p-toluenesulfonic, trifluoroacetic or trifluoromethanesulfonic acid whereas the halide is preferably the chloride.

In a preferred embodiment of the reaction, from 1.8 to 2.0 moles of 4-fluorophenyl magnesium halogenide are used, preferably about 1.8 moles, and from 1.09 to 1.2 moles of 3-dimethylaminopropyl magnesium halogenide, preferably about 1.1 moles, are used for each mole of 5-cyanophthalide.

According to the best mode to carry out the invention, in order to reduce the production of possible undesired by-products, from 1.7 to 1.6 moles of 4-fluorophenyl magnesium halide, preferably about 1.64, are used for each mole of 3-dimethylaminopropyl magnesium halide; this molar ratio can be for example obtained by mixing 3.4 parts by weight of a 20% 4-fluorophenylmagnesium halide solution in said organic solvent and 1 part by weight of a 30% dimethylaminopropylmagnesium chloride solution in said organic solvent.

Moreover, the reaction is carried out in from 1.0 to 1.6 litres of solvent, preferably about 1.2 litres, for each mole of 5-cyanophthalide.

The acid having a pK comprised between 0 and 3, and in particular the orto phosphoric acid, is normally used at a concentration between 55 and 95% by weight, a concentration of about 85% being particularly preferred.

The citalopram is obtained by extraction preferably with toluene/water firstly in an acid environment and then in a basic environment.

On the whole, the invention allows to reduce considerably the reaction and the work-up times, according to a novel and simple way of synthesis. The following examples are for illustration only and are not intended to limit the invention.

### EXAMPLE 1

1) 20% Solution of 4-fluorophenylanagnesium bromide in tetrahydrofuran

53.5 g of magnesium turnings (2.2 mol) and 0.3 g of iodide are charged into a 4-litres reactor at room temperature under nitrogen. The mixture is then heated to 70° C. and a solution of 369.5 g (2.11 mol) of 4-fluorobromobenzene in 1960 ml tetrahydrofuran (5.3 volumes on 4-fluorobromobenzene) is added drop wise, in one hour. After addition the mixture is heated to reflux temperature (68÷70° C.) for 30 minutes, then it's cooled to 25° C.

2000 g of a 20% solution of 4-fluorophenylmagnesium bromide in tetrahydrofuran are obtained (to be kept under nitrogen and protected from the light).

2) 30% Solution of dimethylaminopropyl magnesium chloride in tetrahydrofuran

39.22 g (1.61 mol) of magnesium turnings e 0.3 g of iodide are charged into a 2-liters reactor at room temperature under nitrogen. The mixture is then heated to 70° C. and a solution of 4.53 ml (0.061 mol) of ethylbromide in 72 ml of tetrahydrofuran is added drop wise in 15 minutes. The reaction is quickly seeded. A solution of 208.77 g (1.90 mol) of dimethylaminopropyl chloride in 545 ml of tetrahydrofuran is added drop wise. After addition the mixture is heated to reflux temperature (68÷70° C.) for 30 minutes, then it's cooled to 25° C.

774.1 g of a 30% of dimethylaminopropylmagnesium chloride solution in tetrahydrofuran are obtained (to be kept under nitrogen and protected from the light).

3) Citalopram (Via orto-phosphoric Acid)

A mixture of 1150 g (1.15 mol) of a 20% 4-fluorophenylmagnesium bromide solution and 338 g (0.69 mol) of a 30% dimethylaminopropylmagnesium chloride solution is prepared at room temperature.

The resulting mixture is then added in about 2 hours to a mixture of 100 g (0.63 moles) of 5-cyanophthalide in 750 ml of tetrahydrofuran, under nitrogen at −0÷0° C. After addition (see note 1), 550 ml of orto-phosphoric acid 85% are added drop wise, keeping the temperature below 10° C. After addition the mixture is heated to 66° C. and tetrahydrofuran is distilled; the mixture is then heated to 70÷80° C. for 2 hours. The reaction is finished from HPLC control. 1100 ml of water and 650 ml of toluen are added.

The phases are separated; the aqueous phase is extracted with 200 ml of toluene to give a new aqueous phase.

9 g of active carbon are added to this new aqueous phase; the mixture is stirred for one hour at 25° C., then it's filtered on a supra panel and washed with water (3×50 ml). The filtered is cooled to 5-10° C., 650 ml of toluene are added and about 1300 ml of ammonium hydroxide solution 30% are added till pH 9.5 (keeping the temperature below 15° C.). The possible undissolved salts are filtered washing the panel with toluene (about 200 ml) and the phases are separated. 300 ml of water are added to the organic phase. The phases are separated. The organic phase is evaporated to crude citalopram as an oil. Crude Citalopram: 177 g

4) Citalopram bromhydrate (salyfication)

177 g of crude citalopram obtained from the previous step are dissolved in 300 ml of acetone; it is cooled to 0÷5° C. and about 30 ml of hydrobromic acid 62% are added till pH 1. The suspension is stirred at 0÷5° C. for one hour and the solvent is then evaporated under vacuum. 200 ml of acetone are added and the solvent is evaporated under vacuum at 40° C.; 250 ml of acetone are added and the suspension is stirred at 0÷5° C. for a night. The panel is washed with 3×50 ml of acetone at 0÷5° C. The crude citalopram bromhydrate is dried under vacuum at 60° C.

Crude Citalopram bromhydrate: 95.1 g

5) Citalopram bromhydrate (purification)

95 g of crude citalopram bromhydrate are suspended in 192 ml of deionised water, and the suspension is heated to 60° C. to give a solution. 5.7 g of active carbon are added and the mixture is stirred 30 minutes at 60° C. It's filtered on a supra panel at 60° C. and the panel is washed with deionised water at 60° C. (2×25 ml).

The filtered solution is charged in a 500 ml reactor, it's cooled to 0° C. and it's stirred for a night. On the following day 140 ml of deionised water are added at 0° C. and it's stirred for 5 hours. It's filtered washing the panel with 70 ml of deionised cold water.

The pure citalopram bromhydrate is dried under vacuum at 60° C.

Pure citalopram bromhydrate: 68.8 g (molar yield: 27%; w/w yield: 68.8%).

NOTE 1: At the end of the addition of the mixture of the two Grignards to the suspension of 5-cyanophthalide, an NMR analysis (BRUKER AMX 3-400) is carried out on a sample.

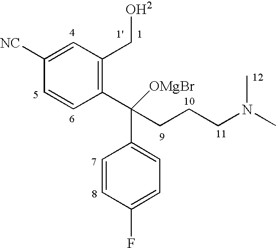

¹H-NMR(DMSO-d6)

| δ(ppm) | Multiplicity | N° H | Attribution |
|---|---|---|---|
| 7.83-7.70 | m | 3 | H4, H5, H6 |
| 7.22-7.05 | m | 4 | H7,H8 |
| 5.1 | s | 1 | H2 |
| 4.52-4.48 | d | 1 | H1 |
| 4.00-3.96 | d | 1 | H1′ |
| 2.98-2.16 | m | 6 | H9,H10,H11 |
| 1.69 | s | 6 | H12 |

This ¹H-NMR is compared to the compound having 2 free hydroxy groups described in EP-171943, here-below reported. By comparison, it's showed that the peak at 6.50 ppm, which corresponds to the hydroxy group in position 3, is lacking in intermediate I.

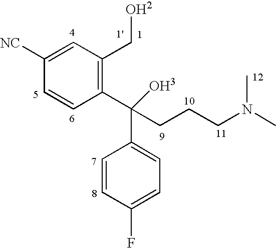

¹H-NMR (DMSO-d6)

| δ(ppm) | Multiplicity | N° H | Attribution |
|---|---|---|---|
| 7.89-7.74 | m | 3 | H4,H5,H6 |
| 7.27-7.07 | m | 4 | H7,H8 |
| 6.50 | s | 1 | H3 |
| 5.16 | s | 1 | H2 |
| 4.60-4.56 | d | 1 | H1 |
| 4.08-4.04 | d | 1 | H1′ |
| 2.29-2.23 | m | 2 | H9 |
| 2.16-2.11 | m | 2 | H11 |
| 2.01 | s | 6 | H12 |
| 1.39-1.19 | m | 2 | H10 |

### EXAMPLE 2

1) Citalopram (Via methanelsulfonyl chloride)

To a suspension of 5-cyanophthalide (100 g, 0.62 mol) in 1500 ml of tetrahydrofuran, a 20% solution of 4-fluorophenylmagnesium bromide in tetrahydrofuran (1100 ml, 1.1 mol), is added under nitrogen, at −5-0° C. Then a 30% solution of dimethylaminopropyl magnesium chloride in tetrahydrofuran (491 ml, 0.96 mol) is added and the reaction stopped by adding a 15% solution of ammonium chloride (1200 ml). After separation, the organic phase is evaporated (50° C.), a 50% mixture of water/toluene (2000 ml) is added and the pH adjusted to 4 by using acetic acid (100 ml). Toluene (1000 ml) is added to the separed aqueous phase and the pH adjusted to 9.3 by adding ammonium hydroxide (170 ml,). To the organic phase triethylamine (470 ml, 3.36 mol) and methanelsulfonyl chloride (47.5 ml, 0.61 mol) in THF is added at −5-0° C. The reaction is stopped by adding water (1200 ml), the solution separated. To the organic phase, water is added (2000 ml) and the pH is adjusted to 5.5 by using acetic acid (230 ml). After separation of the phases, to the aqueous solution toluene is added (1000 ml) and the pH adjusted to 9.3. After extraction, the organic phase is evaporated to obtain crude citalopram as yellow oil (160 g,)

2) Citalopram clorhydrate (salyfication)

16.6 g of crude citalopram obtained from the previous step are dissolved in 44 ml of 1M HCl solution in methanol the pH is adjusted to 1. The solution is evaporated and methylisobutylketone (MIBK) (80 ml) is added, the solution is cooled to 0° C. The solid is filtered off and washed (2*50 ml) with MIBK and acetone (50 ml). Citalopram chlorohydrate is obtained as white solid (8 g).

### EXAMPLE 3

1) Citalopram bromhydrate (via triphenylphosphine)

To a suspension of 5-cyanophthalide (10 g, 0.062 mol) in 150 ml of tetrahydrofuran, a 20% solution of 4-fluorophenylmagnesium bromide in tetrahydrofuran (110 ml, 0.1 mol), is added under nitrogen, at −8° C. Then a 30% solution of dimethylaminopropyl magnesium chloride in tetrahydrofuran (46 ml, 0.09 mol) is added and the reaction stopped by adding a 15% solution of ammonium chloride (120 ml). After separation, the organic phase is evaporated (50° C.), a 50% mixture of water/toluene (200 ml) is added and the pH adjusted to 4 by using acetic acid (10 ml). Toluene (100 ml) is added to the separed aqueous phase and the pH adjusted to 9.3 by adding ammonium hydroxide (17.9 ml). The organic phase is separated to give 17.9 of 2 g di 4-(4-dimethylamine)-1-(4′-fluorophenyl)-1-(hydroxybutyl)-3-(hydroxymethyl)-benzonitrile as a yellow oil. 2 g of 4-(4-dimethylamino)-1-(4′-fluorophenyl)-1-(hydroxybuthyl)-3-(hydroxymethyl)benzonitrile (0.021 mol) are dissolved in 200 ml of THF; 16.54 g of triphenylphosphine (0.0630 mol) are added under vacuum to the solution under stirring. 12.9 ml of ethyl azadicarboxylate (0.081 mol, equivalent to 3.8 mol/mol of starting substrate) dissolved in 50 ml of THF are dropped at 0° C.; 4.83 g of sodium tert-butylate are dropped (0.05 mol, equivalent to 2.5 mol/mol of starting substrate) and the mixture is left overnight. The reaction is stopped by adding 70 ml of a solution of HCl 1N; after evaporation to residue, 150 ml of water and 150 ml of toluene are added and the phases are separated. 150 ml of toluene are added to the aqueous phase and the pH is brought to 9.4 by adding aqueous NH₃ 30%. The phases are separated, the organic phase evaporated, the residue is dissolved in 15 ml of acetone and addede with HBr 62% till a pH of 1. It is filtered yielding 5 g of crude 1-(3-dimethylaminopropyl)-1-(4′-fluorophenyl)-1-3-dihydroxybenzofurane-5-carbonitrile bromhydrate (I, citalopram).

The solid is dissolved in 10 ml of demineralized water, heated and kept at room temperature overnight. 3,5 g 1-(3-dimethylaminopropyl)-1-(4′-fluorophenyl)-1-3-dihydroxybenzofurane-5-carbonitrile bromhydrate (I, citalopram) with a purity of 99.85% as determined by HPLC analysis.

1. A process for the preparation of citalopram characterized in that:
(a) 5-cyanophthalide is treated with a mixture of 4-fluorophenyl magnesium halide and 3-dimethylaminopropyl magnesium halide and (b) the obtained mixture is treated with an organic acid, an inorganic acid, a phosphine, or with a labile ester forming group and a base.

2. A process according to claim 1, characterized by the use of from 1.8 to 2.0 moles of 4-fluorophenyl magnesium halide, for each mole of 5-cyanophthalide.

3. A process according to claim 1, characterized by the use of from 1.09 to 1.2 moles of 3-dimethylaminopropyl magnesium halide, for each mole of 5-cyanophthalide.

4. A process according to claim 1, characterized by the fact that from 1.7 to 1.6 moles of 4-fluorophenyl magnesium halide, are used for each mole of 3-dimethylaminopropyl magnesium halide.

5. A process according to claim 1, characterized by the fact that 4-fluorophenyl magnesium halide is a bromide.

6. A process according to claim 1, characterized by the fact that 3-dimethylaminopropyl magnesium halide is a chloride.

7. A process according to claim 1, characterized by the fact that said acid has a pK comprised from 0 to 3.

8. A process according to claim 1, characterized by the fact that said acid has a pK comprised from 2 to 3.

9. A process according to claim 7, characterized by the fact that said acid is orto-phosphoric acid.

10. A process according to claim 7, characterized by the fact that the acid is used in a concentration comprised from 55 to 95% by weight, preferably in concentration of about 85% by weight.

11. A process according to claim 1, characterized in that the phosphine is thriphenylphosphine.

12. A process according claim 1, characterized in that the labile ester forming group is selected from the halide or the anhydride of an organic acid.

13. A process according to claim 12, characterized in that the halide of the organic acid is the halide of methanesulfonic, p-toluenesulfonic, trifluoroacetic or trifluoromethanesulfonic acid.

14. A process according to claim 13, characterized in that the halide is the chloride.

15. A process according to claim 12, characterized in that base is selected from triethylamine, dimethylaniline or pyridine.

16. A process according claim 1, characterized by the fact that the process is carried out in an organic polar aprotic solvent.

17. A process according to claim 16, characterized by the fact that the process is carried out in from 1.0 to 1.6 litres of solvent, for each mole of 5-cyanophthalide.

18. A process according to claim 16, characterized by the fact that the solvent is selected from tetrahydrofuran and/or toluene.

19. A process according to claim 1, characterized by the fact that the step (a) is carried out at −20÷+20° C.

20. A process according to claim 1, characterized by the fact that the step (a) is carried out at −10÷0° C.

21. A process according to claim 1, characterized by the fact that the step (b) is carried out at −10÷+20° C.

22. A process according to claim 1, characterized by the fact that the step (b) is carried out at 0÷+10° C.

23. A process according to claim 1, characterized by the fact of being carried out without isolating the intermediate products.

24. Compound of formula: 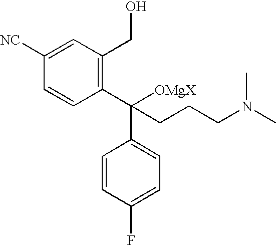
where X is an halogen, preferably chlorine or bromine.

25. Use of a compound according to claim 24 as an intermediate in the preparation of citalopram.
